(19) United States
(12) Patent Application Publication
(10) Pub. No.: US 2003/0093119 A1
(43) Pub. Date: May 15, 2003

(54) Eyelet reinforcement at the tissue-suture interface

(76) Inventors: Chunfeng Zhao, Rochester, MN (US); Peter C. Amadio, Rochester, MN (US); Yu-Long Sun, Rochester, MN (US); Kai-Nan An, Rochester, MN (US); Mark E. Zobitz, Rochester, MN (US)

Correspondence Address: FISH & RICHARDSON P.C., 3300 DAIN RASCHER PLAZA, 60 SOUTH SIXTH STREET, MINNEAPOLIS, MN, 55402, US

(21) Appl. No.: 10/256,998

(22) Filed: Sep. 27, 2002

Related U.S. Application Data
(60) Provisional application No. 60/325,256, filed on Sep. 27, 2001.

(51) Int. Cl. A61L 17/00

(52) U.S. Cl. 606/228

(57) ABSTRACT

The invention relates to a method of strengthening a tissue against the force of a suture by forming a reinforced eyelet in the tissue. Such an eyelet can be formed by any means that results in strengthening or reinforcement of a tissue against the tensile force of a suture. A reinforced eyelet can be formed via the physical attachment of a strengthening means to a tissue or by contacting a tissue with an eyelet-forming agent. Also described are sutures and kits for use with the method of reinforcing a tissue by forming a reinforced eyelet.

### CROSS REFERENCE TO RELATED APPLICATIONS

This application claims priority under 35 U.S.C. §119(e) of U.S. provisional application serial No. 60/325,256, filed Sep. 27, 2001.

### FIELD OF THE INVENTION

The invention is related to methods for repairing tissue, and in particular is related to methods of tissue repair that involve the formation of a reinforced eyelet.

### BACKGROUND

Tendon injuries are a difficult, serious and frustrating problem. Conservative treatment has little, if any, chance of restoring active motion in the affected area. Even after surgical repair, full motion is usually not achieved. Permanent loss of motion, joint contractures, weakness, and stiffness often are unavoidable, despite optimal care. Scar tissue can glue together moving surfaces within the tendon sheath, resulting in adhesions and limited motion. See, Tsuge et al. (1977) J. Hand Surg.—[Am.] 2:436-40; Strickland (1987) Orthop. Rev. 16:137-53; and Manske et al. (1985) Hand Clin. 1:25-34. Repeat surgery can be required to release scar tissue, place tendon grafts, or for other reconstructive procedures. Postoperative therapy includes splinting and physical therapy. Good patient cooperation with such postoperative therapy is essential to obtaining the best possible result. See, Kleinert and Cash (1985) Instructional Course Lectures 34:361-72; Can et al. (1992) J. Hand Surg.—[Am.] 17:1133-9; and Aoki et al. (1997) J. Hand Surg.—[Am.] 22:107-14. In order to permit mobilization immediately after tendon repair, strong suture techniques are necessary to maintain alignment of the lacerated tendon ends until healing occurs. See, Tsuge et al. (1975) Hand 7:250-5; Becker (1978) Hand 10:37-47; Lee (1990) J. Hand Surg. 15A:953-958; and Winters et al. (1997) Annales de Chirurgie de la Main et du Membre Superieur 16:229-34. Excessive loading of the repaired tendon can lead to gap formation or even rupture of the repair. See, Abrahamsson (1991) Scand. J. Plast. Reconstr. Surg. Hand Surg., Supplementum 23:1-51; Silfverskiold et al. (1992) J. Hand Surg.—[Am.] 17:539-46; and Gelberman et al. (1999) J. Bone Joint Surg.—[Am.] 81:975-82. Although numerous suture techniques and various suture materials have been well studied, achieving adequate suture strength remains a surgical challenge due to the small surface area of the tendon. While multiple suture strands can significantly increase the strength of the repair, they also can increase the tendon volume at the repair site. See, Becker (1978) Hand 10:37-47; Savage and Risitano (1989) J. Hand Surg.—[Brit.] 14:396-9; and Winters et al. (1998) J. Hand Surg.—[Am.] 23:97-104. Such increased volume can jeopardize tendon gliding during postoperative therapy due to increased gliding resistance. See, Zhao et al. (1999) Transact. Orthop. Res. Soc. 24:120. A reduction in tendon gliding then can result in increased adhesion formation.

### SUMMARY

The present invention relates generally to the observation that the formation of a reinforced eyelet in tissue increases the holding strength at a tissue-suture interface. A reinforced eyelet can prevent a suture from cutting through the tissue, so that the tendonsuture interface gripping strength increases. Increased gripping strength can prevent gap formation in the sutured tissue and rupture of the surgical repair.

In one aspect, the invention features a method of strengthening a tissue against the force of a suture. The method can involve reinforcing an eyelet in the tissue. An eyelet can be reinforced before passing a suture through the eyelet, coincident with passing a suture through the tissue, or after passing a suture through the tissue. The tissue can be a tendon (e.g., a flexor tendon). The tissue can be a ligament. An eyelet can be reinforced biologically, e.g., by a collagen cross-linking agent such as a carbodiimide. Alternatively, an eyelet can be reinforced chemically, e.g., by a chemical bonding agent such as a cyanoacrylate glue.

In another aspect, the invention features an apparatus for the application of an eyelet-reinforcing agent to a tissue. The invention also features a suture material having an eyelet-reinforcing agent associated therewith.

In yet another aspect, the invention features a kit. The kit can contain a suture material and an eyelet-reinforcing agent. Alternatively, the kit can contain a suture material having an eyelet-reinforcing agent associated therewith. In another embodiment, the kit can contain an eyelet-reinforcing agent.

The invention also features an isolated tendon containing a reinforced eyelet.

In still another aspect, the invention features a method for testing the ability of a compound to reinforce an eyelet. The method can involve: (a) mounting a tissue on a tensiometer, the tissue having the compound applied at the site of an eyelet in the tissue; (b) applying tensile force to a suture loop passing through the eyelet; and (c) measuring the tensile force at which the suture loop is pulled from the tissue.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods and examples are illustrative only and not intended to be limiting.

Other features and advantages of the invention will be apparent from the following detailed description, and from the claims.

### BRIEF DESCRIPTION OF THE DRAWINGS

FIG. 1 is a graph plotting the stiffness of EDC-treated (solid line) and untreated (dashed line) tendons. (N=Newtons; mm=millimeters)

FIG. 2 is a schematic representation of a method for measuring tendon-suture strength using an MTS servohydraulic testing machine. Suture pullout force is measured by pulling on the single loop of the suture.

FIG. 3 is a graph plotting the strength of EDC-treated (solid line) and untreated (dashed line) tendons.

FIG. 4A is a representative cross-sectional drawing of an eyelet in a tendon. FIG. 4B is a representative cross-sectional drawing of the eyelet depicted in FIG. 4A after reinforcement. The stippled portion represents the reinforcement surrounding the eyelet.

FIG. 5 is a graph plotting the average maximum failure force of a single suture loop in tendons containing an eyelet reinforced with EDC compared to the average maximum failure force of a single suture loop in untreated tendons. Ten tendons were tested in each group.

FIG. 6 is graph plotting the average maximum failure force of a single suture loop in tendons containing an eyelet reinforced by local glue injection compared to the average maximum failure force of a single suture loop in untreated tendons. Four tendons were tested in each group.

### DETAILED DESCRIPTION

The invention is based on the discovery that forming a reinforced eyelet in a tissue increases the holding strength of the tissue at a tissue-suture interface. The reinforced eyelet prevents a suture from migrating into the tissue or tearing through the tissue. Gap formation in the sutured tissue and rupture of the surgical repair is minimized or even prevented. This method for reinforcing the edges of a suture hole in tissue against the tensile force of the suture can be used in any procedure in which soft tissue is sutured. Such tissues include, but are not limited to, tendon, ligament, skin, and muscle. The use of such a reinforcing eyelet in surgical tissue repair has many benefits, including increased suture gripping strength without the use of multiple stitches or locking loops. As a result, the complexity of surgical stitching procedures is decreased. With the use of less complex stitching, the mass of the repaired site is reduced. This allows less restricted movement and increased circulation, promoting more rapid healing and decreased adhesion formation.

The model presented is for tendon repair, as this is a common type of repair that fails by suture pullout. However, the concept is applicable to any type of soft tissue suture repair.

Eyelet Formation and Reinforcement

As used herein, an “eyelet” refers to a hole through a tissue (e.g., a tendon). An eyelet can be formed by, for example, inserting a needle through a tissue. A “reinforced eyelet” refers to an eyelet that has been strengthened (e.g., biologically or chemically); the edges of a reinforced eyelet typically are stiffer and/or stronger than the edges of an eyelet that has not been reinforced. A suture placed through a reinforced eyelet can be less likely to tear the tissue surrounding the eyelet than a suture placed through an eyelet that has not been reinforced.

There are a number of means by which a tissue can be reinforced against the tensile force of a suture. For example, an eyelet can be reinforced by the physical attachment of a strengthening means, such as a metal or plastic grommet, to a tissue. An eyelet also can be reinforced by contacting a tissue with an eyelet-reinforcing agent. Such an agent can act either by biologically modifying the tissue itself, or by chemically attaching a bonding agent to the tissue. For example, a cross-linking agent can function as a biological eyelet-reinforcing agent, while a bonding agent such as a glue can function as a chemical eyelet-reinforcing agent. Any means known in the art for applying an agent to a tissue can be used to apply an eyelet-reinforcing agent. For example, an eyelet-reinforcing agent can be spotted on a tissue with, e.g., a needle, stylus, or probe, and allowed to diffuse into the tissue. An eyelet-reinforcing agent can be administered as a bead of liquid present on the tip of a needle as it is passed through a tissue. An eyelet-reinforcing agent can be directly administered into a tissue by, for example, injection through a needle. In another means of delivery, an eyelet-reinforcing agent can be pre-coated on a needle or coated or embedded on a suture material. In this case, the eyelet-reinforcing agent is delivered to the tissue by the act of stitching or suturing. It is noted that such procedures also can be used with surgical staples. In these embodiments, a reinforced eyelet can be formed prior to insertion of the staple, or an eyelet-reinforcing agent can be pre-coated on a surgical staple and thus be delivered to the tissue upon the act of stapling.

Tendon Repair

While the use of a reinforced eyelet is applicable to any type of soft tissue suture repair, the use of reinforced eyelets in tendon repair is presented as a model system, as this is a common type of repair that frequently fails because of suture pullout. The weakest portion of the repaired tendon is the suture-tendon interface. The typical failure mode is of the suture cutting through the tendon causing a gap to form, followed by complete rupture. Some investigators have suggested using multiple or locking loops to increase the suture gripping strength. See, Lin et al. (1988) J. Hand Surg.—[Am.] 13:553-8; Mashadi and Amis (1991) J. Hand Surg. [Brit.] 16:35-9; and Hatanaka and Manske (1999) J. Hand Surg.—[Am.] 24:751-60. However, these procedures can jeopardize the intrinsic blood supply of the tendon, damage the tissues, complicate surgical performance and increase the tendon gliding resistance. See, Lundborg et al. (1977) J. Hand Surg.—[Am.] 2:417-27 and Manske et al. (1984) J. Bone Joint Surg.—[Am.] 66:385-96. The ideal suture construct will have a combination of high strength and low gliding resistance. The present invention provides methods for reinforcing an eyelet in a tissue through which a suture passes, thus increasing the suture gripping strength without the use of multiple or locking loops, and providing a combination of high strength and low gliding resistance.

Structure of Collagen

Collagens are insoluble, extracellular glycoproteins that are found in all animals. Collagen is the most abundant protein in the human body and is an essential structural component of all connective tissues, such as cartilage, bone, tendons, ligaments, fascia and skin. The basic unit of collagen is a polypeptide consisting of the repeating sequence (glycine (Gly)-X-Y)ₙ, where X is often proline and Y is often hydroxyproline. In a collagen molecule, three separate polypeptide chains, each rich in proline and containing a glycine at every third residue, are wound around one another to generate a left-handed triple helix. These collagen molecules are packed together into fibrils. After the fibrils form in the extracellular space, they are greatly strengthened by the formation of covalent cross-links between lysine residues of the constituent collagen molecules. The types of covalent bonds involved are found only in collagen and elastin, giving these molecules enormous tensile strength. If such cross-linking is inhibited, the tensile strength of the fibrils is drastically reduced, collagenous tissues become fragile, and structures such as skin, tendons, and blood vessels tend to tear. See Molecular Biology of the Cell, 3^{rd} Edition, Alberts et al., eds., 1994, Garland Publishing, Inc. (New York, N.Y.), pages 115-116 and 978-984.

Cross-Linking Agents

In one embodiment, the present invention serves to increase suture-holding strength in a tendon and other collagenous tissue by increasing the extent of cross-linking between collagen molecules. Cross-linking the collagen fibers of a tendon to reinforce an eyelet can be accomplished by a number of methods. Cross-linking agents can be selected so as to produce a biocompatible material and can include, without limitation, UV irradiation and chemical cross-linking agents. Suitable chemical cross-linking agents include, for example, acyl-azide, hexamethylene diisocyanate, bisimidates, glyoxal, polyglycerol polyglycidyl ether, adipyl chloride, ribose and other sugars, carbodiimides, and aldehydes such as glutaraldehyde, formaldehyde, and other aldehydes. See, e.g., WO 85/00511.

Carbodiimides include, without limitation, monocarbodiimides and biscarbodiimides. Monocarbodiimides include EDC, cyclohexyl-B-(N-methylmorpholino) ethylcarbodiimide p-toluene-sulfonate (CMC), and 1-(3-dimethylaminopropyl)-3-ethylcarbodiimide methiodide (ETC), for example. Biscarbodiimides include phenylenebis-(ethyl)-carbodiimide, and 1,6-hexamethylenebis (ethylcarbodiimide), for example. Carbodiimides such as EDC can activate carboxyl groups on a collagen molecule, which then can form synthetic peptide bonds with adjacent amino groups, releasing urea. EDC is a well-known monocarbodiimide cross-linking agent, and has been approved for use in human patients.

Bonding Agents

A bonding agent also can serve as an eyelet-reinforcing agent. Suitable bonding agents include, for example, materials that rapidly polymerize when applied to the surface of biological tissue. Such bonding materials include, without limitation, cyanoacrylate-based adhesives, e.g., Eastman 910 and Histoacryl Blue (based on methyl- and butyl 2-cyanoacrylate, respectively), familiar domestic adhesives such as super glue and “CrazyGlue™”, and surgical adhesives. Other suitable bonding agents include fibrin glue and the polymerized compliant tissue sealants discussed in U.S. Pat. No. 6,217,894, for example. Fibrin glue is composed of fibrinogen, blood coagulation factor XIII, and thrombin. See, e.g., U.S. Pat. Nos. 4,627,879, 5,185,001, 5,226,877, 5,290,552, 5,510,102 and 6,083,383. In general, any bonding material can be suitable as a bonding agent provided that it can bond to the biological tissue in the region of the eyelet and can provide the requisite tensile strength.

Suture Material

Suture materials include surgical silk and any other material that can be passed through a tissue during tissue repair (e.g., suturing thread, clips, and staples). To facilitate the delivery of an eyelet-reinforcing agent to the tissue to be repaired, the eyelet-reinforcing agent can be pre-spotted on the suture material in defined positions, or the entire suture material can be pre-coated or embedded with an eyelet-forming agent. In this case, the eyelet-reinforcing agent can be delivered to the tissue surrounding the suture during the act of suturing. The eyelet-reinforcing agent can be pre-coated or embedded on the suture material along with a temperature-sensitive carrier. Such a carrier can remain in a solid state at a temperature less than physiological body temperatures and can liquefy at physiological body temperature, thus facilitating the contact of the eyelet-reinforcing agent with the tissue. An example of such a thermosensitive carrier is a poly(N-isopropylacrylamide) hydrogel. See, e.g., Gutowska et al. (1995) J. Biomed. Mater. Res. 29(7):811-21; Makino et al. (2001) Colloids Surf. B. Biointerfaces 29(4):341-346; and Uludag et al. (2001) Biotechnol. Bioeng. 73(6):510-21.

Kits/Apparatus

In other embodiments, the invention provides kits and articles of manufacture useful for reinforcing an eyelet in a tissue. The kit or article of manufacture can contain suture material and/or an eyelet-reinforcing agent. The suture material can be spotted with an eyelet-reinforcing agent at defined positions, or the entire suture material can be pre-coated or embedded with the eyelet-reinforcing agent. The kit or article of manufacture also can contain an object (e.g., a needle) suitable for forming an eyelet, and in some embodiments, the object can be pre-spotted or pre-coated with an eyelet-reinforcing agent. When the object is a needle with a bore, for example, the eyelet-reinforcing agent can be pre-coated on the outside surface of the needle and/or contained within the bore.

The kit or article of manufacture can contain additional components desirable from a commercial or user standpoint. Components of the kits and articles of manufacture may or may not be sterile. A label or packaging insert indicating the components are to be used for strengthening a tissue against the force of a suture by forming a reinforced eyelet in the tissue with an eyelet-reinforcing agent can accompany the kit or article of manufacture.

The invention also provides an article of manufacture useful for reinforcing an eyelet in a tissue.

The invention also provides an apparatus that can be used in the process of reinforcing an eyelet in a tissue. Such an apparatus can be, for example, a surgical apparatus to facilitate the reinforcement of an eyelet in a tissue, the delivery of an eyelet-reinforcing agent to a tissue surface, or the use of eyelet-tendon repair techniques in arthroscopic surgical procedures.

Isolated Tendons

Eyelet-reinforcing agents can be used to form reinforced eyelets in isolated tendons. Tendons can be obtained from human cadavers or from other species. Isolated tendons, after formation of reinforced eyelets, can be packaged and stored by methods known in the art, such that they are readily available for use in surgical repair procedures.

The invention will be further described in the following examples, which do not limit the scope of the invention described in the claims.

### EXAMPLES

### Example 1

### Cross-Linking a Tendon with EDC

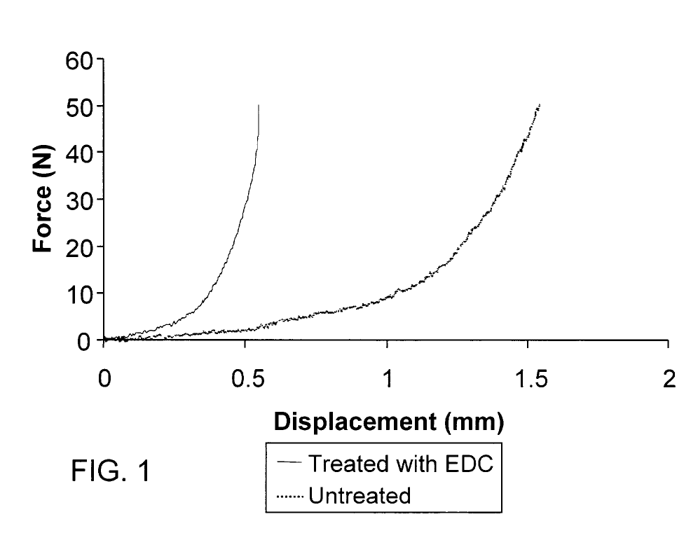

EDC is a cross-linking activating reagent that can couple the carboxyl groups of one collagen molecule to the amino groups of another collagen molecule by forming covalent cross-links. Tendons cross-linked with EDC were much stiffer than untreated tendons. This increase in tendon stiffness was confirmed by studying the mechanical properties of normal and EDC-treated tendons. Each tendon was mounted on a tensiometer, specifically a model 810 MTS servohydraulic testing machine (MTS, Eden Prairie, Minn.) using specially designed clamps. The tendon gage length was approximately 30 mm. A differential variable reluctance transducer (DVRT; MicroStrain, Burlington, Vt.) was attached to a middle portion of the tendon to measure localized tendon elongation. As shown in FIG. 1, the stiffness of a tendon cross-linked with EDC was greater than the stiffness of an untreated tendon. The non-linear portion of the loading curve (toe region) was reduced compared to the normal tendon. Under a 50 Newton load, the tendon within the DVRT portion was elongated about 0.5 mm. Under the same load, the untreated tendon was stretched 1.5 mm, a length three times greater than the tendon treated with EDC. The results indicate that EDC cross-linking yields a tendon that does not stretch easily.

### Example 2

### Measuring Single Suture Loop Failure in EDC-Treated Tendon

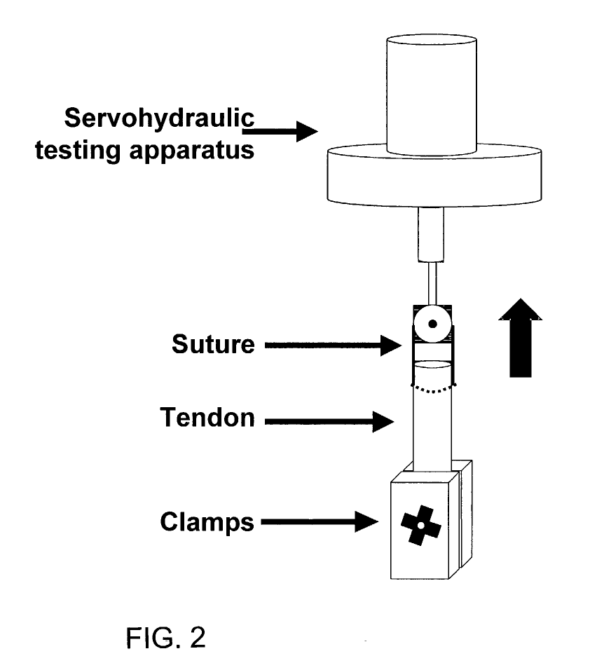

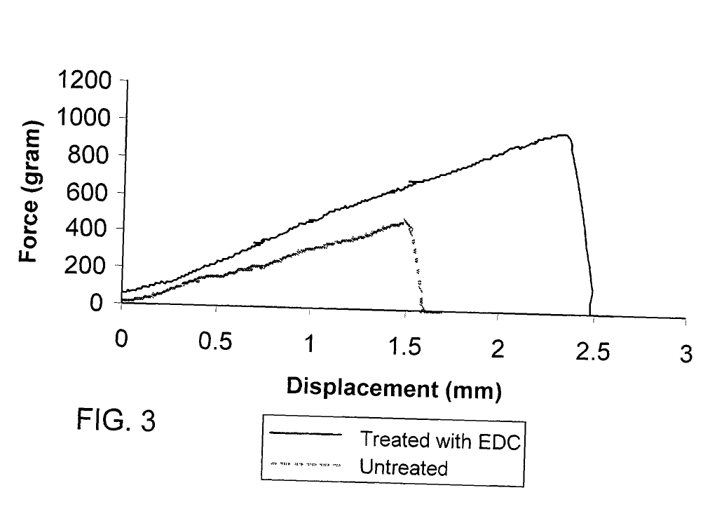

Single loop sutures were passed through both an EDC-treated tendon and an untreated tendon and gripping strength was tested. Tendon specimens were mounted on an MTS servohydraulic testing machine with clamps to secure the tendon, as depicted in FIG. 2. The suture loop was hooked to the movable wheel of the testing machine, and the tendon was distracted at a rate of 20 mm/min until complete suture pullout occurred. Data on tensile force and displacement were collected at a rate of 20 Hz for both EDC cross-linked and untreated tendons. The failure mode of EDC cross-linked tendon was suture breakage at the peak load. However, in the untreated tendons the failure mode was the suture cutting through the tendon until the suture loop pulled out. These results, shown in FIG. 3, indicate that suture strength was dramatically increased by EDC cross-linking of the tendon.

### Example 3

### Measuring Single Suture Loop Failure in Eyelets Formed with EDC

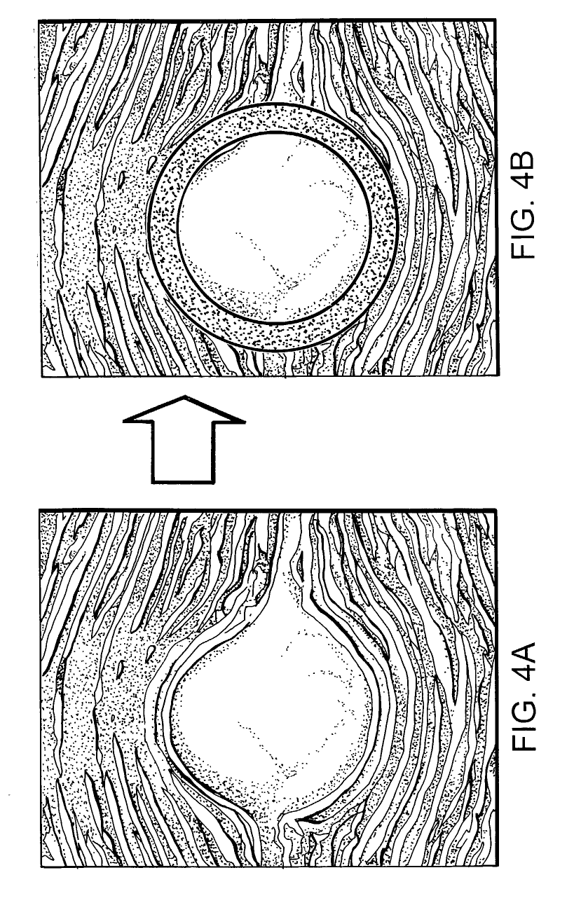

Due to undesirable changes in the overall physiochemical properties of the treated tendon, EDC treatment of an entire tendon is not a practical option for use in tendon repair procedures. Therefore, EDC cross-linking was limited to a region of the tendon immediately adjacent to the suture, in order form a locally reinforced eyelet in the tendon. Such a reinforced eyelet is represented in FIG. 4. Ten flexor tendons from canine forepaws were obtained for testing of the reinforcement. To eliminate the effect of tendon size, each tendon was divided into two pieces. One half of each tendon served as an untreated control, and the other half of the tendon was in the experimental group. A 22-gauge needle was transversely inserted through the tendon 5 mm from the tendon end. Approximately 10 μl of 10% EDC solution was injected into the tendon upon insertion of the needle, so that the EDC was deposited around the needle hole to reinforce the eyelet. 4/0 nylon suture (Ethicon, Somerville, N.J.) was passed through the needle hole, and a single loop suture was placed after the needle was withdrawn. For the control group, saline was injected instead of EDC.

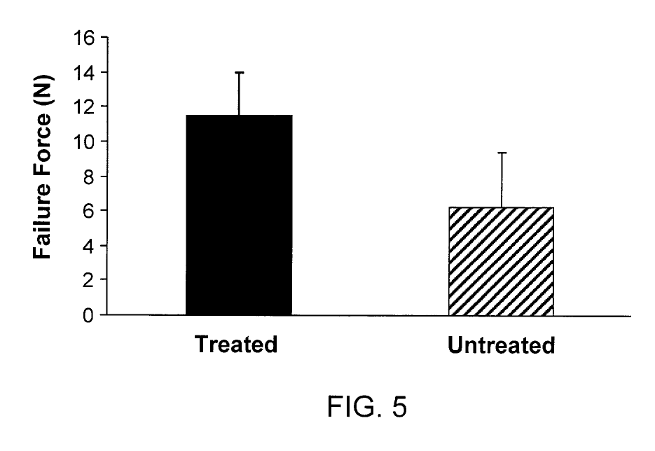

After at least one hour (generally after about one to about six hours), each tendon specimen was mounted on an MTS servohydraulic testing machine with clamps to secure the tendon. The suture loop was hooked to the movable wheel and the tendon was distracted at a rate of 20 mm/min until complete suture pullout occurred. Tensile force and displacement data were collected at a rate of 20 Hz. The failure mode in all untreated tendons and some of the tendons with EDC-eyelets was due to the suture cutting through the tendon. Three tendons in the EDC-eyelet group failed due to suture rupture. As shown in FIG. 5, the average maximum force in the EDC-treated group (11.5±2.4 Newtons) was significantly greater than that of the untreated group (6.2±3.3 Newtons; p<0.001). These results indicate that a reinforced eyelet can withstand a greater force than an unreinforced eyelet, for example, a force that is at least 1.5 times greater (e.g., 1.5 to 2.5 times greater, 1.5 to 3.0 times greater, 2.0 to 3.0 times greater, 1.5 to 4.0 times greater, or more than 4.0 times greater).

### Example 4

### Measuring Single Suture Loop Failure in Eyelets Formed with Superglue

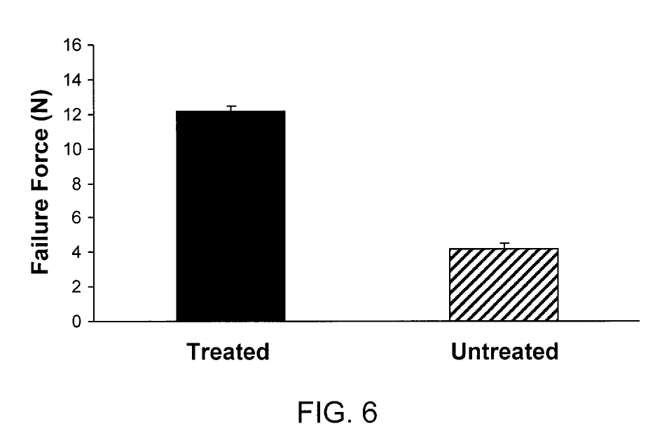

In a further experiment, super glue was applied to tendons to form chemically reinforced eyelets. Three canine flexor digitorum profundus tendons were tested. To eliminate the effect of tendon size, each tendon was divided into two pieces. One half of each tendon served as an untreated control, and the other half of the tendon was in the experimental group. By the same procedures used in Example 3, a 22-gauge needle was used to apply super glue to each tendon to form a reinforced eyelet. A 4/0 nylon suture was then passed through the needle hole, and a single loop suture was placed after the needle was withdrawn. For the control group, saline was injected instead of EDC. Each tendon specimen was mounted on an MTS servohydraulic testing machine with clamps to secure the tendon. The suture loop was hooked to the movable wheel and the tendon was distracted at a rate of 20 mm/min until complete suture pullout occurred. Tensile force and displacement data were collected at a rate of 20 Hz. The average maximum force in the super glue treated group was 12.2 Newtons, while that of the untreated group was 4.2 Newtons (see FIG. 6).

### Example 5

### Effectiveness of the Use of Reinforced Eyelets in Tendon Repair

The effect of a reinforced eyelet on gliding resistance in a repaired tendon can be tested in a canine flexor tendon model using methods such as those outlined in Momose et al. (2000) Appl. Biomater. 53(6):806-11.

The repair of partially lacerated flexor tendon injuries in zone 2 continues to present a challenge to hand surgeons. The repair itself often is detrimental to the biomechanical performance of the flexor tendon, with the additional insult to the tendon caused by suture placement further weakening the overall structure of the tendon. Adhesion formation, suture rupture, and suture locking on the pulley edge are possible consequences of a poor repair. See, Zobitz et al. (2000) J. Biomech. Eng. 122:604. The effectiveness of the use of a reinforced eyelet for repairing partially lacerated tendons can be studied in the partial laceration canine model. Using the methods of Zobitz et al., repair strength (failure load and gap formation) is measured in tendons repaired with reinforced eyelets, and is compared to repair strength in tendons repaired using conventional surgical techniques.

The in vivo effects of reinforced eyelets in surgical tendon repair are determined using methods such as those of Winter et al. and Gelberman et al. For example, tendon strength, gliding function, and range of motion are measured in transected canine flexor tendons repaired in vivo. See, Winters et al. (1998) supra, and Gelberman supra. The compatibility of reinforced eyelets with various suturing techniques is tested by various methods, including those set forth in Lin et al. supra, Winters et al. (1998) supra, Hatanaka and Manske supra, and Zobitz et al. supra.

### Other Embodiments

It is to be understood that while the invention has been described in conjunction with the detailed description thereof, the foregoing description is intended to illustrate and not to limit the scope of the invention, which is defined by the scope of the appended claims. Other aspects, advantages and modifications are within the scope of the following claims.

What is claimed is:

1. A method of strengthening a tissue against the force of a suture, said method comprising reinforcing an eyelet in said tissue.

2. The method of claim 1, wherein said eyelet is reinforced before passing a suture through said eyelet.

3. The method of claim 1, wherein said eyelet is reinforced coincident with passing a suture through said tissue.

4. The method of claim 1, wherein said eyelet is reinforced after passing a suture through said tissue.

5. The method of claim 1, wherein said tissue is a tendon.

6. The method of claim 5, wherein said tendon is a flexor tendon.

7. The method of claim 1, wherein said tissue is a ligament.

8. The method of claim 1, wherein said eyelet is reinforced biologically.

9. The method of claim 8 wherein said eyelet is reinforced by a collagen cross-linking agent.

10. The method of claim 9 wherein said cross-linking agent is a carbodiimide.

11. The method of claim 10 wherein said cross-linking agent is 1-ethyl-3-(3-dimethylaminopropyl) carbodiimide hydrochloride.

12. The method of claim 1, wherein said eyelet is reinforced chemically.

13. The method of claim 12, wherein said eyelet is reinforced by a chemical bonding agent.

14. The method of claim 13 wherein said bonding agent is a cyanoacrylate glue.

15. An apparatus for the application of an eyelet-reinforcing agent to a tissue.

16. A suture material comprising an eyelet-reinforcing agent.

17. The suture material of claim 16, wherein said eyelet-reinforcing agent is a cross-linking agent.

18. The suture material of claim 16, wherein said eyelet-reinforcing agent is a bonding agent.

19. A kit comprising a suture material and an eyelet-reinforcing agent.
